(19) United States
(12) Patent Application Publication
(10) Pub. No.: US 2019/0175772 A1
(43) Pub. Date: Jun. 13, 2019

(54) SYSTEM AND METHOD FOR IN SITU STERILIZATION

(71) Applicant: Pyriscence LLC, Rochester, MI (US)

(72) Inventors: Alexander Donald Blaty, Rochester, MI (US); Thomas Dean Goslinga, San Francisco, CA (US)

(21) Appl. No.: 16/209,758

(22) Filed: Dec. 4, 2018

Related U.S. Application Data
(60) Provisional application No. 62/596,495, filed on Dec. 8, 2017.

(51) Int. Cl. A61L 2/14; A61L 2/24; A61M 25/00

(52) U.S. Cl. CPC A61L 2/14; A61L 2/24; A61L 2202/11; A61L 2202/24; A61M 25/0017

(57) ABSTRACT

An antimicrobial catheter assembly having a catheter tube axially and translatably disposed within a tubular sheath, the sheath penetrating a patient. Embedded within the sheath are anode and cathode wires that may be electrified to generate a sterilizing coronal discharge plasma.

### CROSS-REFERENCE TO RELATED APPLICATIONS

This application is claims the benefit of provisional U.S. patent application Ser. No. 62/596,495, filed. Dec. 8, 2017. The disclosure of the prior application is incorporated herein by reference.

### TECHNICAL FIELD

The several embodiments of this disclosure relate to antimicrobial devices and methods for their sterilization. More particularly, the several embodiments teach systems and methods for sterilizing certain surfaces, and/or adjacent regions of certain surfaces, of a catheter or wound dressing in situ using coronal discharge plasma generation.

### BACKGROUND OF THE INVENTION

Catheters may be useful for several medical applications, including moving, draining, or collecting bod fluids. However, use of catheters may result in complications, such as infections. Catheter-associated infections may result from bacteria being introduced into the body, for example by bacterial adhesion along the exterior (body-facing) surface of the catheter or bacteria migrating along the interior (lumen-facing) surface of the catheter.

Conventional medical sterilization may involve high-pressure steam, ethylene oxide gas, or peroxide gas. These methods may involve high temperatures relative to normal body temperature, use of dangerous or harmful gases ethylene oxide), a vacuum environment, or a prolonged degassing process that may take several days. Therefore, conventional sterilization methods may not be well suited for use in the body (in situ).

Because of these limitations, conventional methods of catheter sterilization may involve removing the catheter from the body, sterilizing the catheter by some means, and reinserting the catheter into the body. Alternatively, the catheter may be removed from the body and replaced with a new sterile catheter. Frequent removal and insertion of catheters into and out of the body of a patient greatly increases the risk of infection to that patient. Moreover, in some medical applications, such as pulmonary artery catheterization (PAC) wherein a catheter is situated within the pulmonary or arterial system, it may be impractical to remove the catheter for sterilization or replacement.

A less conventional medical sterilization method is to utilize coronal discharge plasma generation. This method, sometimes called plasma jet, may be carried out at ambient temperatures and in the atmospheric environment. Although the plasma jet technique has been applied to sterilization of exposed surfaces (surfaces of a medical device outside a patient's body), the technique has significant limitations when applied to catheters in situ, most notably a risk of harming the patient.

To create a plasma jet, plasma may be generated via electric ty in a narrow tube and then discharged through the open end of the tube using a flow of air through the tube. However, utilizing a catheter as the tube to generate a sterilizing plasma jet may be problematic due to the build-up of air and plasma within the body. Further, the air and plasma discharging from the in situ end of the catheter into the body may apply significant forces or force gradients within the body that may physically damage organs or tissues. Additional the air and plasma discharge itself may damage organs or tissues by plasma-induced cellular death or particulate contamination due to improperly filtered air.

Catheter-related infections can be a major cause of prolonged hospitalization. Further, significant antibiotic use associated with treating catheter-related infections can be a major cause of the ongoing and increased antibiotic resistance of certain infectious agents. This necessitates the development of a technology that permits a catheter to be sterilized while within a patient's body.

This disclosure teaches systems and methods for sterilizing a catheter or part a catheter that is inside the body, part of a catheter that is outside the body and adjacent to a part of the catheter that inside the body, or parts of a catheter assembly or un such as a sheath in a Swan-Ganz catheter, using coronal discharge plasma generation. Coronal discharge plasma act to sterilize the surface of the catheter and possibly also adjacent body surfaces or body cavities.

### BRIEF SUMMARY OF THE INVENTION

The several embodiments of this disclosure relate to a catheter or catheter assembly that can be sterilized in situ via plasma generated by coronal discharge. A coronal discharge is an electrical discharge caused by the ionization of a gas or liquid adjacent o an electrically charged object. The object, called an electrode, may be any conductor (or semiconductor or insulator) capable of accepting charged particles. The terms conductor and electrode may be used interchangeably in this disclosure. Further, the charged particles may be electrons, but they may also be molecular or atomic particles or ions having positive or negative charge.

In one embodiment, one or more pairs of conductors may be embedded into the catheter wall one conductor being an anode and the other conductor being a cathode. In another embodiment, one or more pairs of conductors may be embedded in a sheath surrounding a catheter external and adjacent to the point of entry of the catheter into the body. In another embodiment, one or more pairs of conductors may be embedded in a bandage, gauze, or dressing external and adjacent to the point of entry of the catheter into the body, or proximal to an incision, port, wound or opening into the body.

A voltage potential may be applied across the anode-cathode pair. When the distance between the conductors is sufficiently small, and/or the voltage between the conductors is sufficiently high, a large electric field may be created between the conductors that may ionize the air between and adjacent to the conductors. This ionization is a coronal discharge, which may sterilize the air, catheter surfaces, and body surfaces adjacent to the plasma. The applied voltage may be AC or DC depending on, for example, the desired cycle time or intensity of coronal discharges.

### BRIEF DESCRIPTION OF THE DRAWINGS

The drawings presented are for illustrative purposes only of selected embodiments and do not depict all possible embodiments or implementations thereof. The drawings are not intended to limit the scope of the present disclosure.

FIG. 1 is perspective view of a first embodiment.

FIG. 2 a cross-sectional view of the first embodiment taken along line A-A of FIG. 1 (transverse to a longitudinal axis of the catheter).

FIG. 3 is a cross-sectional view of a second embodiment taken transverse to a longitudinal axis of the catheter.

FIG. 4 is a cross-sectional view of a third embodiment taken transverse to a longitudinal axis of the catheter.

FIG. 5 is a cross-sectional view of a fourth embodiment taken transverse to a longitudinal axis of the catheter.

FIG. 6 is a side view of a fifth embodiment (parallel to a longitudinal axis of the catheter).

FIG. 7 is cross-sectional view of the fifth embodiment taken along line B-B of FIG. 5 (transverse to a longitudinal axis of the catheter).

FIG. 8 is cross-sectional view of the fifth embodiment taken along line C-C of FIG. 6 (transverse to a longitudinal axis of the catheter).

FIGS. 9a-9c are perspective views of a sixth embodiment.

FIG. 10 is a cross-sectional view of a seventh embodiment.

### DETAILED DESCRIPTION OF THE INVENTION

The following embodiments are exemplary and the concepts associated therewith may be embodied in various and alternative forms. Some features may be exaggerated or minimized to show details of particular components. Therefore, specific functional or structural details disclosed herein are not to be interpreted as limiting in any way but merely as representative of these particular disclosed embodiments with the understanding that these may vary with respect to other exemplary embodiments.

The following numerals are used to identify the corresponding elements in the figures for the several embodiments.
10 catheter
20 catheter assembly
30 bandage
100 cathode
102 anode
104 catheter lumen
106 catheter wall
108 patient tissue
110 catheter exterior surface
112 catheter interior surface
114 patient body cavity
116 insulating material
118a,b anode-cathode pair
120 conductive mesh
122 secondary catheter
200 balloon
300 atmospheric space
302 apex
400 contamination sheath
410a,b collar
420 introducer sheath
500 bandage interior
510 bandage surface

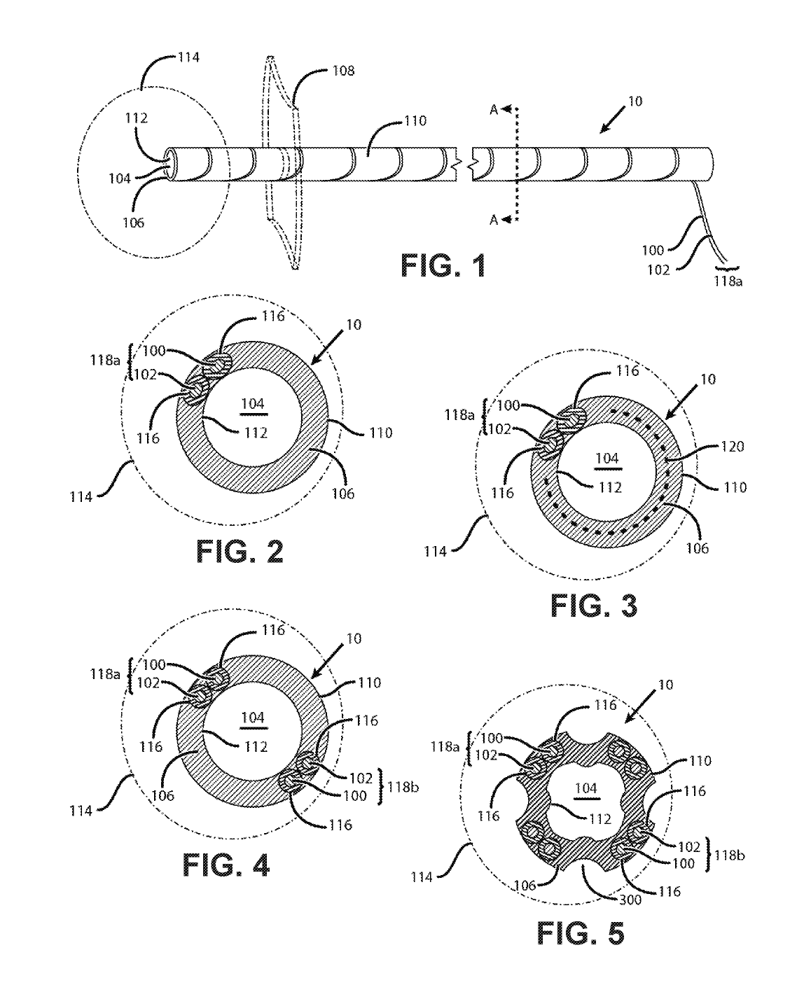

FIG. 1 shows a catheter 10 having a catheter wall 106 defined by an exterior surface 110 and an interior surface 112. The catheter 10 and/or catheter lumen 104 may have any cross-sectional shape whether circular, oval, smooth, lagged, or otherwise (see FIG. 5 for an example of a non-circular catheter 10). A lumen 104 is contained within the space created by a circumference of the interior surface 112. Fluid may flow through the catheter lumen 104 or any other space external to or along the catheter exterior surface 110. This fluid may be bodily fluid that naturally flows through the patient body cavity 114 or any other type of fluid injected into the patient body cavity 114 such as saline.

A cathode 100 and/or an anode 102 may be embedded on or within the catheter wall 106. Alternatively, one conductor may be embedded on or within the catheter wall 106, for example the cathode 100, and the other conductor may be formed by the patient's body, for example the anode 102 (not illustrated). The catheter wall 106 may be electrically insulating, or alternatively or in addition, the cathode 100 and/or the anode 102 may be encased within a layer of insulating material 116. The insulating material 116 may provide additional electrical insulation around the cathode 100 and/or the anode 102.

While the catheter 10 is in use, the catheter 10 may be positioned within a patient body cavity 114 that may be defined by a cavity wall of patient tissue 108. A voltage potential may be applied to the cathode 100 and/or anode 102. Alternatively, an electric current may be driven into or out of the cathode 100 and/or anode 102. The voltage or electric current may be AC or DC, having fixed or variable amplitude, frequency, or phase (if multiphase). The cathode-anode naming convention of the conductors is not meant to a particular conductor to a conventional polarity, for example the cathode 100 may be electrically charged positively or negatively and the anode 102 may be electrically charged negatively or positively, respectively.

The cathode 100 and the anode 102 may be disposed adjacent to each other as shown in FIGS. 1-5, or the cathode 100 and the anode 102 may be disposed on substantially opposite sides of the catheter lumen 104 (not illustrated). The cathode 100 and the anode 102 may spiral around the catheter lumen 104 as a pair as shown in FIG. 1; they may spiral around the catheter lumen 104 independently (e.g., in opposite directions); they may run substantially parallel with the catheter lumen 104 as indicated in FIGS. 4-5; or they may be oriented in an arbitrary manner.

FIG. 3 shows an optional conductive mesh that may be embedded within (or on) the catheter wall 106. The conductive mesh 120 may be electrically coupled to either the cathode 100 or the anode 102, or the conductive mesh 120 may serve as a ground plane.

An adjacent cathode 100 and anode 102 may form an anode-cathode pair 118a as shown in FIGS. 1-5. There may be multiple anode-cathode pairs 118, for example an anode-cathode pair 118a and an anode-cathode pair 118b as shown in FIGS. 4-5. The number of anode-cathode pairs 118 may be based on a number of factors, for example the circumference or wall thickness of the catheter 10, the intended use or ease-of-use of the catheter 10, or the desired manufacturing cost. Configuring the cathode 100 and the anode 102 into pairs or wires may help prevent intermingling of these wires to provide simpler manufacturing or more efficient electrical properties of the catheter 10. Each anode-cathode pair 118 may be driven by the same or a separate power supply that may or may not share a common ground or a common phase.

One advantage of configuring the cathode 100 and the anode 102 adjacent to each other is to reduce the distance between the cathode 100 and the anode 102. This may increase the electric field (volts/meter) between the cathode 100 and the anode 102 for a given applied voltage between the cathode 100 and the anode 102. A high electric field may be beneficial to generating a plasma-inducing coronal discharge. Consequently, for patient-safety reasons it may be desirable to induce a coronal discharge using a lower voltage applied to a closely separated cathode 100 and anode 102 rather than using a higher voltage applied to a distantly separated cathode 100 and anode 102. Additionally, a lower voltage may increase the electrical efficiency of the system.

A voltage potential applied or induced between the cathode 100 and the anode 102 may create a sufficiently large electric field gradient to ionize the air or matter between and adjacent to the cathode 100 and the anode 102. The voltage and/or current applied to or induced upon the cathode 100 and the anode 102 may be AC or DC to achieve a desired cycle time or intensity of coronal discharges. This ionization may lead to a coronal discharge, which may in turn create plasma capable of causing significant cellular death of bacteria or microorganisms (bacteriocidal), or which may sufficiently damage bacteria and microorganisms to inhibit or prevent their reproduction (bacteriostatic), while causing no or minimal harm or damage to adjacent patient tissue 108. Consequently, the air, catheter exterior surface 110, catheter interior surface 112, patient body cavity 114, and/or the patient tissue 108 may be effectively and safely sterilized.

Some catheters have a smooth outer surface in which there are few if any air gaps between the patient tissue 108 and the catheter exterior surface 110. However, because plasma may be more efficiently generated by coronal discharge in dry or gaseous conditions compared to moist or fluidic conditions, it may be desired to create air pockets on the catheter exterior surface 110. This may be achieved in several ways, for example by a textured, corrugated, dimpled, or ribbed segment of catheter exterior surface 110. This uneven segment of the catheter exterior surface 110 may extend along some or all of the length of the catheter 10 and it may have variably dimensioned peaks, troughs, and gaps. The catheter exterior surface 110 may or may not contact patient tissue 108.

FIG. 5 shows a catheter 10 having a longitudinally corrugated catheter exterior surface 110 such that an atmospheric space 300 exists between adjacent anode-cathode pairs 118. (FIG. 5 shows four anode-cathode pairs 118 but only two are labeled).

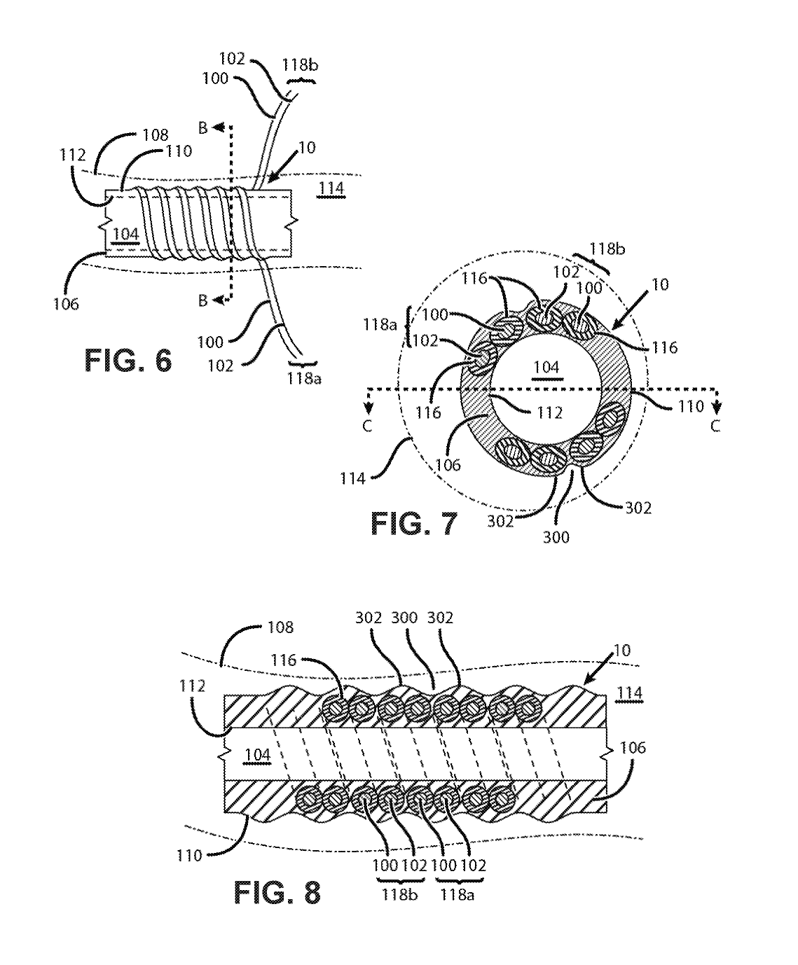

FIGS. 6-8 shows a catheter 10 having a ribbed (helical) catheter exterior surface 110 (FIG. 6 is a side view, FIG. 7 is a transverse cross-sectional view, and FIG. 8 a close-up longitudinal cross-sectional view). An atmospheric space 300 exists between each pair of adjacent apexes 302 of the ribbing on the catheter exterior surface 110.

Additionally, atmospheric space 300 may be created by modulating the chemical or physical properties of the catheter wall 106. For example, microscopic atmospheric space 300 may be created when part or all of the catheter wall 106 comprises a permeable material.

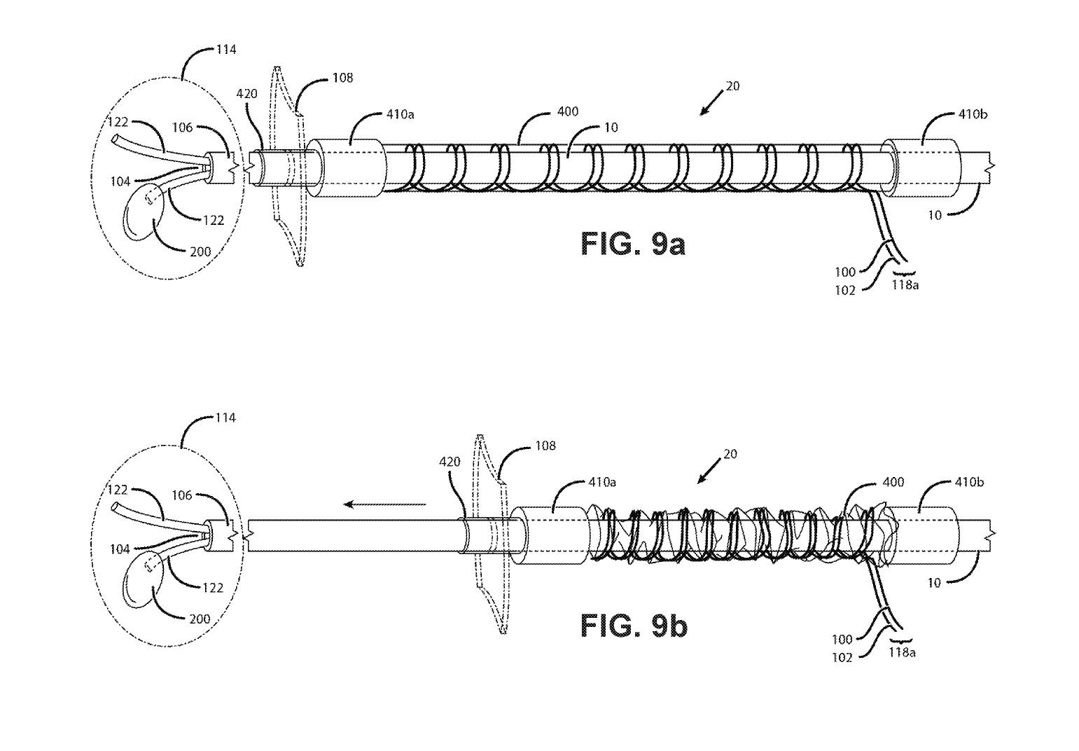

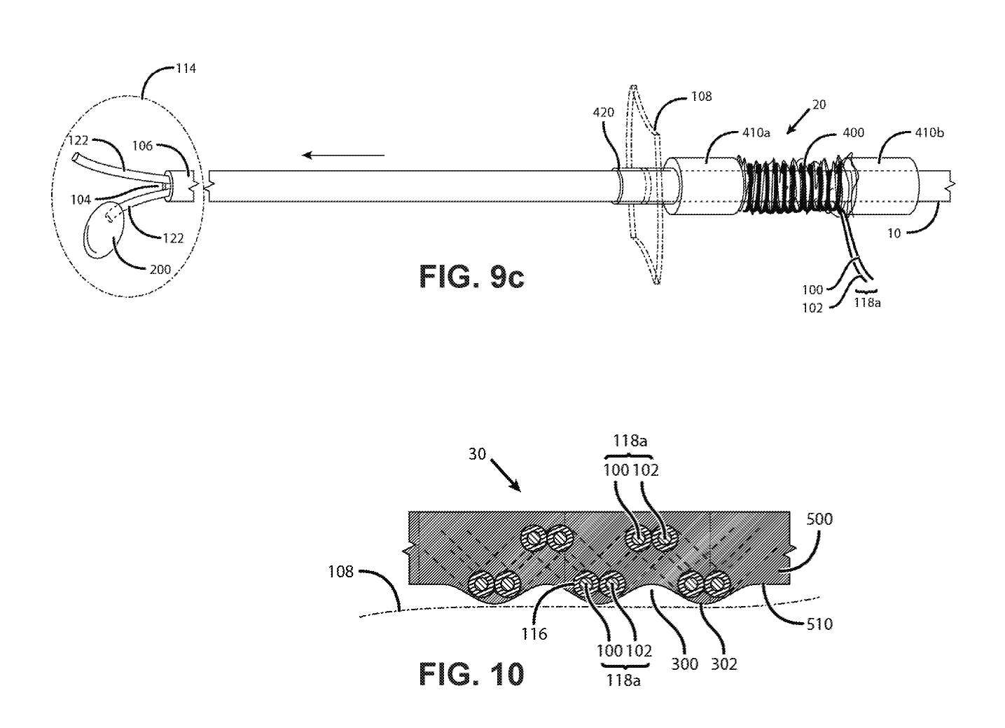

FIGS. 9a-9c show a catheter assembly 20 that may be used in a pulmonary artery catheterization (PAC) medical procedure, sometimes called Swan-Ganz catheterization. The catheter assembly 20 comprises a catheter 10 that may enter the patient body through an introducer sheath 420. The introducer sheath 420 may be attached to a first tubular collar 410a that is adjacent to the patient tissue 108. A second tubular collar 410b may be coupled to the first collar 410a by means of a tubular contamination sheath 400 that may be flexible or rigid. The catheter 10 is disposed axially, through the first collar 410a, the second collar 410b, and the contamination sheath 400. The contamination sheath 400 may be composed of a suitable plastic, rubber, or other elastomeric material. FIGS. 9a-9c depict the contamination sheath 400 as being transparent.

One or more secondary catheters 122, instrumentation wires, sensor leads, fiber-optic cables, conduits, channels, or other sensors, tools, or implements may be disposed within the primary catheter lumen 104. For example, FIGS. 9a-9c show a secondary catheters 122 that terminates into a balloon 200 that may be inflated within a patient body cavity 114 or adjacent to a patient organ.

The catheter 10 may be advanced into or withdrawn from the patient body by axial translation through the contamination sheath 400 and through one or both of the first collar 410a and the second collar 410b. A purpose of the contamination sheath 400 is to maintain sterility of the catheter 10 during and between successive advancements and withdrawals. The contamination sheath 400 may, but need not be, be flexible, compressible, extendable, elastic, pliant, or foldable (longitudinally pliant similar to an accordion). This helps prevent a segment of atmosphere-exposed catheter 10 from entering the contamination sheath 400 during advancement of the catheter 10 and/or helps prevent a segment of non-atmosphere-exposed catheter 10 from within the contamination sheath 400 to be exposed to the atmosphere during withdrawal of the catheter 10. However, even with a longitudinally pliant contamination sheath 400, is possible to translate a potentially non-sterile segment of the catheter 10 (atmosphere-exposed) into the contamination sheath 400 though the second collar 410b, as shown in FIG. 9b, which may introduce bacteria and microorganisms to the interior cavity of the contamination sheath 400. When this occurs, subsequently translating a segment of the catheter 10 out from the contamination sheath 400 though the first collar 410a and into a patient, as shown in FIG. 9c, may introduce those bacteria and microorganisms into the patient body.

FIG. 9a shows a cathode 100 and the anode 102 (comprising an anode-cathode pair 118a) embedded within the contamination sheath 400. One or both of the cathode 100 and the anode 102 may be disposed on or within the contamination sheath 400, or alternatively one both of the cathode 100 and the anode 102 may be disposed on or within the catheter 10 as illustrated in previous figures. Additionally, there may be multiple anode-cathode pairs 118 as previously discussed. The cathode 100 and the anode 102 may spiral about the catheter 10 as shown in FIGS. 9a-9c. Alternatively, they may ran parallel to the catheter 10 or they may be oriented in an arbitrary manner as indicated in the discussion of previous embodiments.

FIG. 9b shows the catheter 10 having been translated into the contamination sheath 400 through the second collar 410b (also the contamination sheath 400 has been compressed longitudinally and is therefore illustrated as having wrinkles). To kill any bacteria or microorganisms that may have been carried into the contamination sheath 400, the cathode 100 and/or the anode 102 may be electrified to generate a sterilizing coronal discharge plasma as previously described. Therefore, a segment of the catheter 10 that may be subsequently translated out from contamination sheath 400 through the second collar 410b and into the patient body, as shown in FIG. 9c, is sterile.

Due to the accordion-like behavior of the contamination sheath 400, it may have many folds and wrinkles. This can make it difficult to fully sterilize the interior of the contamination sheath 400 using conventional methods such as by injecting a sterilizing fluid or gel. Such a fluid or gel may not reach all the folds and creases within the contamination sheath 400 (and it may also impede the translation of the catheter 10 by clogging the openings in the collars 410 or by drying out). In contrast, sterilization via coronal discharge plasma generated by conductors embedded within or adjacent to the contamination sheath 400 may more easily kill bacteria and microorganisms hiding in these folds and wrinkles. Moreover, these folds and wrinkles, combined with the generally dry environment within the contamination sheath 400, create air pockets that may increase the efficiency of generating sterilizing coronal discharge plasma as previously discussed.

FIG. 10 shows a bandage 30 wherein the cathode 100 and the anode 102 are embedded within a material comprising the bandage interior 500. The cathode 100 and/or the anode 102 may be encased in an insulating material 116 as shown, or they may be embedded in one or more dielectric layers within the bandage interior 500 (not illustrated). In the case wherein the cathode 100 and the anode 102 are defined by electrical wires, such wires may be routed, woven, layered, or disposed on or within the bandage interior 500 in any suitable manner that may enable coronal discharge upon sufficient electrification thereof. Alternatively, one conductor may be embedded on or within the bandage interior 500, for example the cathode 100, and the other conductor may be formed by the patient's body, for example the anode 102 (not illustrated).

The bandage surface 510 may be positioned adjacent to patient tissue 108 to dress a wound, for example. The bandage surface 510 may create atmospheric spaces 300 adjacent to the patient tissue 108 by means of a texture, corrugation, dimpling, or ribbing of the bandage surface 510 defining a plurality of apexes 302. As previously described, a voltage and/or current may be applied to the cathode 100 and/or anode 102 of sufficient magnitude to induce a sterilizing coronal discharge.

The foregoing embodiments are exemplary and should not be interpreted as limiting the scope of the several embodiments. Various implementations and combinations of these embodiments have been recognized and anticipated. In some cases, features of embodiments can be combined in different ways and still achieve desirable results. Additionally, the several embodiments depicted in the accompanying figures do not necessarily require the particular depiction shown to achieve desirable results.

What is claimed is:

1. An antimicrobial catheter, comprising:
(a) a first tube having a wall, a first region outside a patient, and a second region inside the patient, the second region including an uneven exterior surface defining a plurality of air pockets;
(b) at least one conductive electrode embedded within the wall of the second region generally along the axis of the first tube, each electrode having an electrical contact on the first region;
(c) a power source capable of coupling a voltage to the electrical contact of each electrode, the voltage being of sufficient magnitude to create a coronal discharge adjacent to at least one electrode.

2. The catheter of claim 1 wherein the uneven exterior surface comprises corrugations, ribs, dimples, or textures having peaks and valleys.

3. The catheter of claim 1 further comprising at least one conduit axially disposed within the lumen of the first tube.

4. The catheter of claim 3 wherein the conduit is a channel to transmit information from a sensor within the patient.

5. The catheter of claim 4 wherein the channel comprises an electrical wire or a fiber-optic cable.

6. The catheter of claim 3 wherein the conduit comprises a second tube coupled to an inflatable balloon inside the patient.

7. An antimicrobial catheter, comprising:
(a) a tubular first sheath axially joining a tubular first collar to a tubular second collar;
(b) a tubular second sheath axially attached to the first collar opposite the first sheath, the second sheath penetrating a patient;
(c) a first tube, having a lumen, axially and translatably disposed within the first sheath, the second sheath, the first collar, and the second collar;
(d) at least one conductive electrode embedded within a wall of the first sheath generally along the axis of the sheath, each electrode having an electrical contact;
(e) a power source capable of coupling a voltage to the electrical contact of each electrode, the voltage being of sufficient magnitude to create a coronal discharge adjacent to at least one electrode.

8. The catheter of claim 7 wherein the first sheath is longitudinally pliant.

9. The catheter of claim 7 further comprising at least one conduit axially disposed within the lumen of the first tube.

10. The catheter of claim 9 wherein the conduit is a channel to transmit information from a sensor within the patient.

11. The catheter of claim 10 wherein the channel comprises an electrical wire or a fiber-optic cable.

12. The catheter of claim 9 wherein the conduit comprises a second tube coupled to an inflatable balloon inside the patient.

13. A method for sterilizing a catheter in situ, comprising:
(a) advancing a catheter tube into a patient through a tubular sheath that penetrates the patient;
(b) applying a voltage potential to at least one electrode embedded within a segment of the sheath outside the patient sufficient magnitude to create a coronal discharge adjacent to the electrode.

14. The method of claim 13 wherein the sheath comprises a generally rigid region proximal to the location of penetration and a longitudinally pliant region outside the patient.

15. An antimicrobial bandage, comprising:
(a) a sheet having a thickness, a first surface, and a second surface defining a plurality of air pockets adjacent to a patient tissue;
(b) at least one conductive electrode embedded within the thickness of the sheet, each electrode having an electrical contact on the first surface;
(c) a power source capable of coupling a voltage to the electrical contact of each electrode, the voltage being of sufficient magnitude to create a coronal discharge adjacent to at least one electrode.

16. The bandage of claim 15 wherein the uneven exterior surface comprises corrugations, ribs, dimples, or textures having peaks and valleys.

17. The bandage of claim 15 further having a catheter penetrating therethrough, the catheter having a first region outside a patient and a second region inside the patient.
